(19) United States
(12) Patent Application Publication
(10) Pub. No.: US 2006/0074049 A1
(43) Pub. Date: Apr. 6, 2006

(54) Use of heparinoids for treatment and prevention of dementia

(76) Inventors: Mitchell Krathwohl, Maple Grove, MN (US); Jodi L. Anderson, Minneapolis, MN (US); Walter C. Low, Shorewood, MN (US)

Correspondence Address: FISH & RICHARDSON P.C., PO BOX 1022, MINNEAPOLIS, MN, 55440-1022, US

(21) Appl. No.: 10/958,556

(22) Filed: Oct. 5, 2004

(51) Int. Cl. A61K 31/737; A61K 31/727

(52) U.S. Cl. 514/54; 514/56

(57) ABSTRACT

Methods for treating, preventing, or reducing development of non-traumatic interneuron pathologies are described herein. Also described are methods for treating or preventing dementia (e.g., dementia associated with HIV, AIDS, or Alzheimer's disease).

### STATEMENT AS TO FEDERALLY SPONSORED RESEARCH

Funding for the work described herein was provided in part by the National Institutes of Health, grant number AI01544. The federal government may have certain rights in the invention.

### TECHNICAL FIELD

This invention relates to methods for treating subjects having non-traumatic interneuron pathologies, and more particularly to methods for treating and preventing dementia.

### BACKGROUND

Proliferating neural progenitor cells (NPC) in the adult human are capable of differentiating into both astrocytes and neurons (Roy et al. (2000) J. Neurosci. Res. 59:321-331; Palmer et al. (2001) Nature 411:42-43; and Roy et al. (2000) Nat. Med. 6:271-277). These newly formed neurons are thought to subsequently form synaptic connections with other neurons (Kruger et al. (2002) Cell 110:399-402). One area of the brain in which new neuron formation is thought to play a functional role is the dentate gyrus of the hippocampus (Fuchs and Gould (2000) Eur. J. Neurosci. 12:2211-2214). New neurons within the hippocampus are thought to increase memory capacity and replace neurons lost through aging (Kempermann (2002) J. Neurosci. 22:635-638). Additional evidence suggests that many NPC exist in a quiescent state, apparently awaiting future needs in the host (Morrison et al. (1997) Cell 88:287-298; and Bonfanti et al. In: Stem cells and CNS development, Rao M., ed., Humana Press, Totowa, N.J., 2001:370). It has recently been shown that human NPC can be forced into quiescence by the constitutively produced chemokine SDF-1 and the inflammatory chemokines RANTES and eotaxin (Krathwohl et al. (2004) Stem Cells 22:109-118). Quiescence induction by SDF-1 is mediated by the CXCR4 receptor, while quiescence induced by RANTES and eotaxin is mediated by the CCR3 receptor.

### SUMMARY

The invention is based in part on the discovery that human immunodeficiency virus (HIV) can inhibit NPC proliferation. In particular, HIV coat proteins that use the chemokine receptors CCR3 or CXCR4 as co-receptors can inhibit proliferation of NPC in isolated cultures and in hippocampal slices. In addition, cerebrospinal fluid from HIV patients with dementia can inhibit NPC proliferation in these culture systems. Moreover, hippocampal tissue from HIV patients with dementia at autopsy contained reduced numbers of NPC as compared to the numbers of NPC in hippocampal tissue from non-demented patients. Thus, there is an in vivo correlation between HIV dementia and NPC proliferation.

The invention also is based in part on the discovery that apolipoprotein E3 can compete for binding to cell surface heparan sulfate (HS) on NPC, and can antagonize the effects of HIV coat proteins on NPC proliferation. This finding led the inventors to show that HS (formulated as danaparoid, for example) can result in increased proliferation of NPC in vivo. Thus, the invention provides compositions and methods for stimulating NPC proliferation and for treating, preventing, or reducing the development of non-traumatic interneuron pathologies such as, for example, dementia (e.g., HIV-induced dementia).

In one aspect, the invention features a method for treating a patient having a non-traumatic interneuron pathology. The method can include administering to the patient a heparinoid, wherein the heparinoid is administered in an amount effective to reduce the symptoms of the non-traumatic interneuron pathology.

In another aspect, the invention features a method for treating a mammal. The method can include administering to the mammal a heparinoid, wherein the heparinoid is administered in an amount effective to stimulate proliferation of NPC. The method can further include monitoring the cognitive function of the mammal. In addition, the method can further include adjusting the amount of heparinoid administered to the mammal based on the monitoring.

In another aspect, the invention features a method for treating dementia in a mammal in need thereof. The method can include administering to the mammal an effective dose of a heparinoid. The mammal can be a human. The mammal can be HIV positive. The mammal can have AIDS. The mammal can be diagnosed with Alzheimer's disease. The heparinoid can be heparan sulfate (e.g., heparan sulfate that is at least 70 percent pure, at least 90 percent pure, or at least 99 percent pure). The heparan sulfate can be in combination with chondroitin sulfate. The heparinoid can be danaparoid.

In still another aspect, the invention features a method for preventing dementia in a mammal. The method can include administering a heparinoid to the mammal. The mammal can be a human. The mammal can be HIV positive. The mammal can have AIDS. The mammal can be diagnosed with Alzheimer's disease. The heparinoid can be heparan sulfate (e.g., heparan sulfate that is at least 70 percent pure, at least 90 percent pure, or at least 99 percent pure). The heparan sulfate can be in combination with chondroitin sulfate. The heparinoid can be danaparoid.

The invention also features methods for treating or preventing memory loss in a mammal. The methods can include administering to the mammal an effective dose of a heparinoid.

In yet another aspect, the invention features a method for treating spinal cord injury in a mammal. The method can include administering a heparinoid to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing the number of NPC colonies displaying outgrowth of cells with neuronal morphology after treatment with coat proteins from the HIV strains 93TH975, CM235 and IIIb. The mean+/−standard error is shown. PTX=pertussis toxin, Anti V3=monoclonal antibody to the V3 loop region of gp120. FIG. 1B is a series of graphs showing the level of bromodeoxyuridine (BrdU) incorporation into NPC neurospheres treated with coat proteins from the HIV strains 93TH975, CM235 and IIIb. Shaded area=control cells, solid line=treated cells.

FIG. 2A is a graph showing the level of ERK phosphorylation in NPC treated with coat proteins from the HIV strains 93TH975, CM235, and IIIb. Cells were cultured with the coat proteins in the absence or presence of the phosphatase inhibitor okadaic acid (OA). Mean+/−standard error is shown. FIG. 2B is a series of graphs showing the number of p-ERK expressing cells in NPC cultures incubated with and without the indicated HIV coat proteins.

FIG. 3A is a graph showing the number of colonies formed after incubation of NPC with cerebrospinal fluid (CSF) from demented and non-demented HIV. Each point represents the average of triplicate values for one patient. Demented+Ab=a broadly neutralizing monoclonal antibody to HIV was added to CSF from demented patients prior to addition to NPC cultures; Fraction <30 kD=CSF filtered to remove molecules>than 30,000 daltons; Isotype=irrelevant antibody of same isotype as neutralizing antibody. FIG. 3B is a graph showing the correlation of viral load with number of colonies of differentiating NPC treated with CSF from demented HIV patients. FIG. 3C is a graph showing the correlation of viral load with number of colonies of differentiating NPC treated with CSF from non-demented HIV patients.

FIG. 4A is a graph showing the total number of BrdU positive NPC in hippocampal slice cultures incubated with the indicated HIV coat proteins. Mean+/−standard error is shown. OA=okadaic acid; Ab=broadly neutralizing antibody to V3 region of gp120. FIG. 4B is a graph showing the number of BrdU positive cells in hippocampal slice cultures incubated with CSF from control subjects or from HIV patients with and without dementia.

FIG. 5 is a graph showing the number of Ki-67 positive cells in hippocampal samples from autopsied control subjects and HIV patients with and without dementia.

FIG. 6 is a graph showing the number of BrdU labeled NPC in samples treated with recombinant HIV gp120 from the IIIb strain, which was pre-incubated with or without recombinant ApoE3 and ApoE4.

### DETAILED DESCRIPTION

HIV infection can cause cognitive decline, a syndrome generally referred to as HIV dementia. Dementia is a general term for a decline in a person's intellectual function. Demented patients typically are awake and alert, but can have varying degrees of cognitive impairment. The exact mechanism by which HIV produces dementia is unknown.

The inventors have observed that HIV coat proteins that use the chemokine receptors CCR3 or CXCR4 as co-receptors can inhibit proliferation of NPC in isolated cultures and in hippocampal slices. In addition, cerebrospinal fluid from HIV patients with dementia can inhibit NPC proliferation in these culture systems. Moreover, hippocampal tissue from HIV patients with dementia at autopsy contained reduced numbers of NPC in demented patients as compared to non-demented patients. Thus, there is an in vivo correlation between HIV dementia and NPC proliferation.

The invention also is based in part on the discovery that apolipoprotein E3 can compete for binding to cell surface HS on NPC, and can antagonize the effects of HIV coat proteins on NPC proliferation. This finding led the inventors to show that HS (e.g., formulated as danaparoid) can result in increased proliferation of NPC in vivo. Thus, the invention provides compositions and methods for treating, preventing, or reducing the development of non-traumatic interneuron pathologies such as, for example, dementia (e.g., HIV-induced dementia).

1. Compositions

Heparinoids are compounds related to heparin, an anticoagulant. Compositions that are useful in the methods provided herein can contain a heparinoid such as, for example, HS or dermatan sulfate. Heparin is not considered a heparinoid. When included within a composition for treating or reducing non-traumatic interneuron pathologies, a heparinoid such as HS can be at least about 50% pure (e.g., at least about 60%, about 65%, about 70%, about 75%, about 80%, about 85%, about 90%, about 95%, about 98%, about 99%, or about 100% pure). That is, a composition can contain at least about 50% HS (e.g., at least about 60%, about 65%, about 70%, about 75%, about 80%, about 85%, about 90%, about 95%, about 98%, or about 99%, or can be 100% HS). In some embodiments, a composition can contain about 70% to about 85% HS in combination with other ingredients such as chondroitin sulfate (CS). For example, danaparoid is a low molecular weight heparinoid that is derived from porcine gut mucosa, and typically contains about 70% HS in combination with CS as well as dermatan sulfate. Danaparoid (also referred to as Orgaran®) is an antithrombotic agent that has been used to prevent deep venous thrombosis during and after surgery, for example. Since danaparoid is devoid of heparin or heparin fragments, it also has been used as an alterative therapy in patients who develop thrombocytopenia in response to treatment with heparin.

Heparinoids such as HS and danaparoid are commercially available. HS can be obtained from Sigma Chemical Co. (St. Louis, Mo.) as a sodium salt, for example. Danaparoid can be obtained from Orgaron (Roseland, N.J.). In some embodiments, commercially available forms of HS can be further purified (e.g., using chromatography such as anion exchange or gel filtration chromatography) prior to use in the methods described herein.

Methods for formulating and subsequently administering compositions are well known to those skilled in the art. Dosing typically is dependent on the severity and responsiveness of the condition to be treated, with the course of treatment lasting from several days to several months, or until a cure is effected or a diminution of the condition is achieved. Persons of ordinary skill in the art are able to determine optimum dosages, dosing methodologies and repetition rates. Optimum dosages can vary depending on the relative potency of individual compounds, and can be estimated based on EC₅₀ found to be effective in in vitro, ex vivo, and/or in vivo animal models. Dosages also can vary with the route of administration, the nature of the condition being treated, and the age and condition of the subject to be treated. Typically, dosage is between about 0.01 μg and about 100 g per kg of body weight (e.g., between about 0.1 μg and about 50 g per kg of body weight, between about 0.5 μg and about 25 g per kg of body weight, or between about 1 μg and about 10 g per kg of body weight). Dosages may be given once or more daily, weekly, or even less often. Following successful treatment, it may be desirable for a to patient undergo maintenance therapy to prevent recurrence of the condition.

The present invention provides pharmaceutical compositions and formulations that include a heparinoid such as HS. HS therefore can be admixed, encapsulated, conjugated or otherwise associated with other molecules, molecular structures, or mixtures of compounds such as, for example, liposomes, receptor targeted molecules, or other formulations for assisting in uptake, distribution, and/or absorption.

A compound such as HS can be combined with a pharmaceutically acceptable carrier (also referred to as an “excipient”), which is a pharmaceutically acceptable solvent, suspending agent, or any other pharmacologically inert vehicle for delivering one or more compounds to a subject. Pharmaceutically acceptable carriers can be liquid or solid, and can be selected with the planned manner of administration in mind so as to provide for the desired bulk, consistency, and other pertinent transport and chemical properties, when combined with one or more of compounds and any other components of a given pharmaceutical composition. Typical pharmaceutically acceptable carriers that do not deleteriously react with heparinoids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

Pharmaceutical compositions can be formulated for administration by a number of methods, depending upon whether local or systemic treatment is desired and upon the area to be treated. Administration can be, for example, parenteral, topical, pulmonary, or oral. Compositions and formulations for parenteral, intrathecal or intraventricular administration can include sterile aqueous solutions, which also can contain buffers, diluents and other suitable additives (e.g., penetration enhancers, carrier compounds, and other pharmaceutically acceptable carriers). Formulations for topical administration can include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be necessary or desirable. Compositions and formulations for oral administration can include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the compound to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

Compositions useful in the methods provided herein can further encompass any pharmaceutically acceptable salts, esters, or salts of such esters, or any other compound which, upon administration to an animal (e.g., a human), is capable of providing (directly or indirectly) the biologically active metabolite or residue thereof. Accordingly, for example, the invention provides pharmaceutically acceptable salts of HS, prodrugs and pharmaceutically acceptable salts of such prodrugs, and other bioequivalents. The term “prodrug” indicates an agent that is prepared in an inactive form and is converted to an active form (i.e., drug) within the body or cells thereof by the action of endogenous enzymes or other chemicals and/or conditions. The term “pharmaceutically acceptable salt” refers to a physiologically and pharmaceutically acceptable salt of a compound (i.e., a salt that retains the desired biological activity of the parent heparinoid without imparting undesired toxicological effects). Examples of pharmaceutically acceptable salts of heparinoids include, but are not limited to, salts formed with cations (e.g., sodium, potassium, calcium, or polyamines such as spermine), acid addition salts formed with inorganic acids (e.g., hydrochloric acid, hydrobromic acid, sulfuric acid, phosphoric acid, or nitric acid), salts formed with organic acids (e.g., acetic acid, citric acid, oxalic acid, palmitic acid, or fumaric acid), and salts formed from elemental anions (e.g., chlorine, bromine, and iodine).

Pharmaceutical compositions containing a heparinoid such as HS also can incorporate penetration enhancers that promote the efficient delivery to the skin. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether), fatty acids (e.g., oleic acid, lauric acid, myristic acid, palmitic acid, and stearic acid), bile salts (e.g., cholic acid, dehydrocholic acid, and deoxycholic acid), chelating agents (e.g., disodium ethylenediaminetetraacetate, citric acid, and salicylates), and non-chelating non-surfactants (e.g., unsaturated cyclic ureas).

In some embodiments, a pharmaceutical composition containing one or more heparinoids also can contain one or more other agents that function by a non-anticoagulant mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions of the invention. Other non-antisense agents (e.g., chemotherapeutic agents) also are within the scope of this invention. Such combined compounds can be used together or sequentially.

Compositions useful in the methods provided herein additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, a composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the heparinoid component(s) within the compositions.

The pharmaceutical formulations of the present invention, which can be presented conveniently in unit dosage form, can be prepared according to conventional techniques well known in the pharmaceutical industry. Such techniques include the step of bringing into association the active ingredients (e.g., one or more heparinoids) with the desired pharmaceutical carrier(s) or excipient(s). Typically, a formulation can be prepared by uniformly bringing the active ingredient(s) into intimate association with liquid carriers or finely divided solid carriers or both, and then, if necessary, shaping the product. Formulations can be sterilized if desired, provided that the method of sterilization does not interfere with the effectiveness of the heparinoid(s) contained in the formulation.

Compositions can be formulated into any of many possible dosage forms such as, without limitation, tablets, capsules, liquid syrups, soft gels, suppositories, and enemas. The compositions of the present invention also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

2. Methods

The three major classes of neurons are sensory neurons, motor neurons, and interneurons. Sensory neurons run from the various types of stimulus receptors (e.g., touch, odor, taste, sound, and vision) to the central nervous system. The cell bodies of sensory neurons leading to the spinal cord are located in clusters, called ganglia, next to the spinal cord. The axons usually terminate at interneurons. Motor neurons transmit impulses from the central nervous system to the muscles and glands that carry out the response. Most motor neurons are stimulated by interneurons, although some are stimulated directly by sensory neurons.

Interneurons, also referred to as “connector neurons” or “association neurons,” are found exclusively within the spinal cord and brain, and are stimulated by signals reaching them from sensory neurons and/or other interneurons. Interneurons connect only with other neurons and not with either sensory cells or muscles, and are thus involved in the intermediate processing of signals.

The invention provides methods for treating, preventing, or reducing development of interneuron pathologies (e.g., non-traumatic interneuron pathologies). As used herein, an “interneuron pathology” is a condition that affects interneurons without significantly affecting sensory neurons or motor neurons. A “non-traumatic” pathology is a condition brought about by a non-traumatic event (e.g., a condition that results from an illness rather than a head injury, for example). As used herein, a stroke (e.g., an ischemic stroke or a hemmorhagic stroke) is not considered to be a non-traumatic pathology. Interneuron pathologies can include, without limitation, conditions such as memory loss or dementia. The term “dementia” is used to describe symptoms that occur when the brain is affected by specific diseases and conditions. Dementia can arise as a result of illnesses such as HIV, AIDS, Alzheimer's disease, and a variety of other conditions as described below. Symptoms of dementia (which also is referred to as “cognitive impairment”) can include forgetfulness, concentration problems, language difficulties, problems with short term memory, clumsiness, unsteadiness, jerky eye movements, ataxia (“drunken gait”), personality changes, loss of appetite, inappropriate emotional responses, mood swings, and hallucinations, for example.

Causes of dementia include, without limitation, Alzheimer's disease, vascular disease, Lewy bodies, and fronto-temporal dementia (including Pick's disease), as well as many other conditions. Alzheimer's disease is the most common cause of dementia. Vascular disease can result in reduced oxygen supply to the brain, which can cause brain cell death. The symptoms of vascular dementia can occur either suddenly, following a stroke, or over time, through a series of small strokes. Dementia with Lewy bodies gets its name from tiny spherical structures that develop inside nerve cells. Their presence in the brain leads to degeneration of brain tissue, affecting memory, concentration, and language skills. In fronto-temporal dementia, damage is usually focused in the front part of the brain. Personality and behavior initially are more affected than memory. Rarer causes of dementia include, for example, progressive supranuclear palsy, Korsakoff's syndrome, Binswanger's disease, Creutzfeldt-Jakob disease (CJD), and HIV or AIDS. People with multiple sclerosis, motor neuron disease, Parkinson's disease, and Huntington's disease also can be at an increased risk of developing dementia.

Methods of the invention can be used to treat or prevent any type of dementia. In one embodiment, the methods provided herein can be used for treating or preventing dementia associated with HIV or AIDS. Dementia due to HIV also is referred to as AIDS dementia complex, HIV-associated dementia, HIV/AIDS encephalopathy, HIV-associated cognitive/motor complex, subacute HIV encephalitis, HIV-associated dementia complex, AIDS-related dementia, and HIV dementia.

Methods provided herein also can be used to stimulate proliferation of NPC in a subject. For example, a subject having an interneuron pathology such as dementia may benefit from increased proliferation of NPC. A method can include administering to a subject a heparinoid in an amount that is effective to stimulate proliferation of NPC, thus increasing the number of NPC within the subject. An amount that is effective can be determined from in vitro or ex vivo studies, as discussed above. An “increase” in the number of NPC can be any increase in the number of NPC (e.g., a 5%, 10%, 15%, 20%, 25%, 30%, 40%, 50%, 60%, 70%, 75%, 80%, 85%, 90%, 95%, 100%, or more than a 100% increase in the number of NPC) compared with the number of NPC prior to treatment, for example. Methods for measuring NPC numbers or NPC proliferation include, for example, NPC colony formation (i.e., level of quiescence) and labeling with BrdU, as described in the Examples herein.

Methods provided herein include administration of an effective dose of a heparinoid to a subject (e.g., a mammal such as a human, a dog, a cat, a non-human primate, a horse, or a cow) in need thereof. An “effective amount” of a compound is an amount that is effective to increase NPC proliferation, as discussed above, to an amount that is effective to reduce the symptoms of a particular condition (e.g., dementia) or to prevent, delay or reduce development of symptoms of the condition in a subject at risk thereof. Typically, the effectiveness of a heparinoid can be monitored by evaluating the cognitive function of the subject using known techniques. When the subject is a human, for example, a mental status examination (e.g., Folstein's Mini-Mental State Examination) can be used to measure the subject's overall mental health. This type of examination is a standard test that can be used by healthcare professionals to evaluate, for example, a subject's state of consciousness (alertness and orientation), memory, and attention span/concentration, visual processes, language, and executive functions (e.g., inhibition, planning, time perception, internal ordering, working memory, self-monitoring, verbal self-regulation, motor control, regulation of emotion, and motivation). A subject's cognitive abilities can be evaluated before and after treatment with a heparinoid, to determine whether such treatment results in improved cognitive function. In some cases, the subject may be able to recognize the effectiveness of a heparinoid simply due to an improvement in their mental capacity. If the subject is a research animal, other techniques can be used to assess cognitive function such as memory and learning. For example, the Morris water maze task is one example of a method that can be used to evaluate cognitive function in rodents. In non-human primates, more sophisticated memory and learning tests can be used (see, e.g., the methods described in Herndon et al. (1997) Behav. Brain Res. 87:25-34).

A composition containing one or more heparinoids (e.g., HS) can be administered to a subject via any suitable route including, for example, parenterally (e.g., by subcutaneous, intrathecal, intraventricular, intramuscular, or intraperitoneal injection, or by intravenous drip), topically (e.g., transdermal, ophthalmic, or intranasal), pulmonarily (e.g., by inhalation or insufflation of powders or aerosols), or orally. Administration can be rapid (e.g., by injection) or can occur over a period of time (e.g., by slow infusion or administration of slow release formulations). For treating tissues in the central nervous system, heparinoids can be administered by injection or infusion into the cerebrospinal fluid, preferably with one or more agents capable of promoting penetration across the blood-brain barrier. In some embodiments, administration of a heparinoid can be combined with another treatment such as, for example, administration of highly active antiretroviral immunotherapy in the case of HIV or AIDS.

The methods provided herein can further include a step in which the cognitive function of the heparinoid treated mammal is evaluated. Methods that can be used to assess cognitive function include those described above, for example. Cognitive function can be assessed prior to treatment and/or at any time following the onset of heparinoid treatment. Thus, cognitive function can be evaluated prior to administration of an initial dose of a heparinoid, after administration of a first dose, or after administration of a plurality of doses (e.g., after 2, 3, 4, 5, or more than 5 doses). Cognitive function can be monitored once (e.g., once after administration of one or more doses of a heparinoid) or more than once (e.g., prior to administration of the first dose and again after administration of one or more doses of a heparinoid, or after administration of a first dose and again after administration of a subsequent dose of a heparinoid).

In some embodiments, a method can further include adjusting the dose of heparinoid administered based on the monitoring of cognitive function. For example, if the cognitive function of a treated mammal does not show signs of improvement, or if symptoms of dementia are observed after one or more heparinoid treatments, the dose of heparinoid can be increased. Alternatively, if the cognitive function of a treated mammal shows significant improvement, or if no symptoms of dementia are observed, the dose of heparinoid can be reduced.

### EXAMPLE 1

### Materials and Methods

Cells and Materials: Normal human NPC were obtained from Clonetics Corp (San Diego, Calif.), and grown in neural progenitor maintenance medium (Clonetics) with human recombinant basic fibroblast growth factor, epidermal growth factor, neural survival factors and antibiotics. These cells were derived as described previously (Svendsen (1998) J. Neurosci. Meth. 85:141-152).

Recombinant full-length monomeric coat proteins produced in a Baculovirus expression system were obtained from Immunodiagnostics, Inc. (Woburn, Mass.), except for CM235, which was obtained from Protein Sciences Corp. (Meriden, Conn.), and 93TH975, which was obtained from the AIDS Research and Reference Reagent Program at AnorMed, Inc. (Langley, British Columbia). Recombinant apolipoproteins E3 and E4 were obtained from PanVera Corporation (Madison, Wis.). AMD3100 was obtained from the AIDS Research and Reference Reagent Program.

Banked CSF samples from clinic visits or autopsy were obtained from the California NeuroAIDS Tissue Network (CNTN; San Diego, Calif.). These samples had been characterized at the CNTN as to patient HIV viral load, CD4 count, neuropsychiatric ratings, and CSF viral load. Neuropsychological scores were assessed as previously described (Masliah et al. (1997) Ann. Neurol. 42:963-972) and patients were rated on a 9-point scale. Patients with scores in the average range (1 to 3) were considered non-demented, and patients with scores of 6-9 were considered demented. For some experiments, CSF was filtered through a size-exclusion column (Millipore, Bedford, Mass.) to remove proteins larger than 30,000 Daltons. RANTES was able to pass through this column into the fraction less than 30,000 Da and inhibit NPC in the plating assay. A cross-reactive neutralizing murine monoclonal antibody to the V3 loop of gp120 was obtained from Immunodiagnostics, Inc. Archived tissue samples of hippocampus from autopsied HIV patients were obtained from CNTN.

Plating Assay: On day 0, HIV coat proteins at a concentration of 800 pM, or CSF at a concentration of 2% was added to cultures of NPC. Each protein or CSF sample was tested in triplicate. Cultures were incubated for seven days, and then cells were plated onto 24 well polyethylene-imine coated plates. The number of cell colonies having more than 20 cells with typical neuronal morphology was counted for each well after 7 days in culture. Determinations of colony inhibition for all proteins and CSF samples were done on each of three different lots of NPC. Each lot was passaged 1-3 times before testing, with no differences seen between this number of passages.

Hippocampal slice cultures: Discarded hippocampal tissue was obtained from patients undergoing corrective surgery for seizures as previously described (Roy et al. (2000) Nat. Med. 6:271-277; and Pincus et al. (1998) Neurosurg. 42:858-868). Tissue was obtained within 1 hour of removal and cultured as described (Stoppini et al. (1991) J. Neurosci, Meth. 37:173-182). Briefly, tissue was sliced to 200 μm thickness using a Brendel-Vitron tissue slicer (Vitron, Inc., Tucson, Ariz.). Slices were placed in a Transwell insert (Costar, Corning, N.Y.) and cultured in neural progenitor maintenance medium (NPMM) with or without chemokines (500 ng/ml), HIV coat proteins (1 nM) or CSF (2%) added on day 0. Each sample was tested in triplicate. The entire experiment was performed at least twice with identical results. Slices were labeled with BrdU on day 5 of culture, and harvested for immunohistochemistry on day 7 by freezing slices in OCT. Five-micron sections were made, and BrdU incorporation was detected using a BrdU in situ kit (Pharmingen, San Diego, Calif.). For dual staining of BrdU and Musashil, a polyclonal antibody to Musashil (Chemicon International, Temecula, Calif.) was used at a dilution of 1:10. Apoptotic cells were detected using a TUNEL assay (Oncogene, Boston, Mass.).

Immunohistochemistry: Archived samples of hippocampus from HIV patients were deparaffinized and incubated with a 1:10 dilution of a mouse monoclonal antibody to Ki-67 (Pharmingen). A secondary biotinylated antibody to mouse IgG was then incubated with the sample, and detection was performed using an avidin-horseradish peroxidase-DAB staining kit (Pharmingen). The number of cells with clearly stained nuclei in 5 high power fields in the subgranular zone of the dentate gyrus was determined for 5 separate sections from each patient, and totaled.

For p21 and p27 detection, NPC were grown with recombinant coat proteins for 24 hours. Cells were fixed and monoclonal antibodies to p21 or p27 (Pharmingen) were added. A secondary antibody conjugated to Cy3 was used for detection.

Signaling: Isolated NPC were incubated with coat proteins for 10 minutes, placed on ice, fixed, permeabilized with saponin, and stained with fluorescein isothiocyanate-(FITC-) conjugated phosphospecific monoclonal antibodies to ERK (Santa Cruz Biotechnology, Santa Cruz, Calif.). Cells were then analyzed by flow cytometry. Activity of PP2A was inhibited by incubation with okadaic acid (Calbiochem, San Diego, Calif.) at 2.5 μM for 1 hour prior to the addition of coat proteins. Experiments were performed at least twice, and determinations were performed in triplicate.

Statistics: Results were analyzed using the JMP 4.0 statistical program (SAS Institute, Cary, N.C.) using one-way ANOVA testing followed by Student's t test. Flow cytometry was analyzed using the FlowJo 4.0 program (Tree Star, Inc., San Carlos, Calif.).

### EXAMPLE 2

### HIV Coat Proteins can Induce Quiescence in Human NPC

The ability of HIV coat proteins to induce quiescence in human NPC was tested using NPC in the form of neurospheres. Typically, neurospheres with quiescent cells fail to attach and differentiate, while neurospheres containing proliferating cells will attach and differentiate into neurons and astrocytes. To determine whether only those coat proteins that signal through either the CXCR4 receptor or the CCR3 receptor would induce quiescence of NPC, purified recombinant coat proteins from several different strains of HIV were tested. These included the R5 strain CM235, the X4 strain IIIb, and the clade E/A recombinant R5 strain 93TH975 (Gao et al. (1996) J. Virol. 70:1651-1667). NPC were incubated with the coat proteins at a concentration of 800 pM for 7 days and then plated on polyethylene-imine coated plates. The number of colonies containing more than 20 cells with typical neuronal morphology was counted after 7 days in culture. In these experiments, both CM235 and IIIb coat proteins caused NPC to enter a quiescent state and fail to attach to the coated plates, while the 93TH975 coat proteins had no effect. None of the coat proteins induced cell death in NPC, as determined by trypan blue staining (>95% viability in all coat-protein treated cultures). To confirm that quiescent cells in CM235 and IIIb-treated cultures maintained viability and the ability to differentiate, the non-attached cells were washed and replated on coated plates. These cells were capable of resuming proliferation, attaching to coated plates, and differentiating.

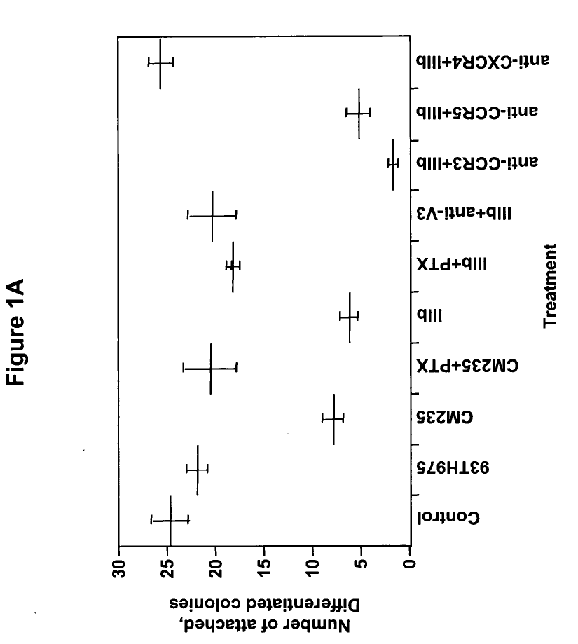

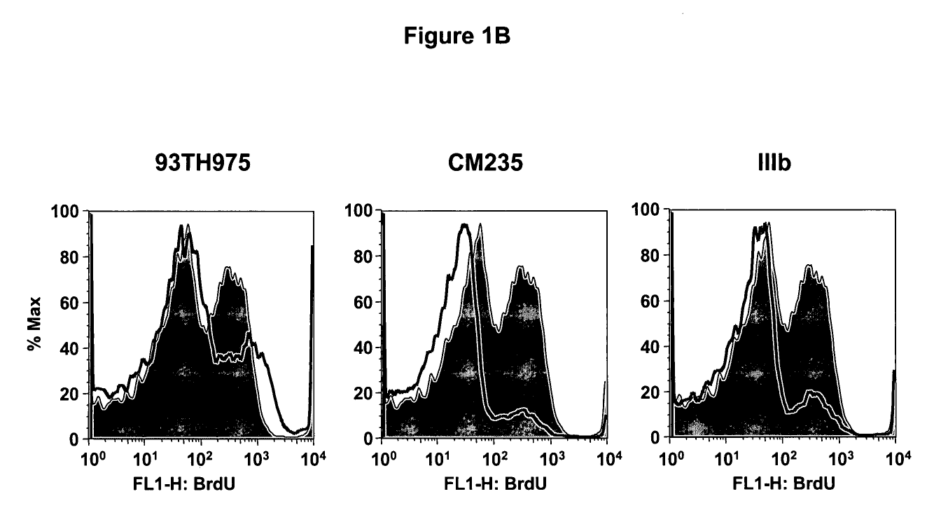

To quantitate the ability of the coat proteins to cause cells to become quiescent, the number of attached, differentiated colonies was counted. These studies revealed that the CM235 and IIIb coat proteins reduced the number of differentiated NPC colonies by 67% and 74%, respectively (p<0.001 for both, FIG. 1A). In contrast, the 93TH975 coat protein had no effect. In order to confirm that cell proliferation was indeed inhibited by the coat proteins, cultures of NPC were labeled with BrdU, and BrdU incorporation was measured by flow cytometry. There was less BrdU incorporation into NPC treated with IIIb and CM235 coat protein than in controls (FIG. 1B), whereas there was no difference in BrdU incorporation in 93TH975 treated cultures as compared to controls. This inhibition of BrdU incorporation was transient, as both IIIb and CM235 treated cells showed incorporation similar to controls when tested 7 days after addition of coat proteins.

To further confirm the involvement of coat proteins in quiescence induction, the effect of coat proteins on expression of the cyclin-dependent kinase inhibitors p21 and p27 was examined. NCP were incubated with 800 pM recombinant coat proteins, and expression of p21 and p27 was analyzed using immunohistochemistry. These experiments revealed that both the CM235 and IIIb coat proteins induced expression of p21 and p27. The induction of both p21 and p27 by the IIIb coat protein was blocked by the CXCR4 receptor antagonist AMD3100. Thus, certain HIV coat proteins were able to inhibit proliferation of NPC and induce cellular quiescence.

### EXAMPLE 3

### Effects of HIV Coat Proteins are Mediated by Chemokine Receptor Binding

Experiments were conducted to evaluate whether the suppressive effects of HIV coat proteins are mediated by chemokine receptor binding. Chemokine receptors are coupled to pertussis toxin-sensitive G-proteins (Rollins (1997) Blood 90:909-928). Thus, pertussis toxin was added to cultures of NPC in addition to HIV coat proteins. The inhibitory effects of both the CM235 and the IIIb coat proteins were eliminated by pertussis toxin (FIG. 1A). Next, the effect of a monoclonal antibody specific for the chemokine-binding region of the HIV coat protein, the V3 loop, was evaluated. These studies showed that the anti-V3 antibody also blocked the inhibitory action of the IIIb coat protein. In addition, the ability of monoclonal antibodies to the CXCR4, CCR3 and CCR5 receptors to block the effects of the IIIb coat protein was tested. In these experiments, the CXCR4 antibody abolished the inhibitory effect of the IIIb coat protein, while the CCR5 and CCR3 antibodies failed to do so. These results suggest that the suppressive effects of HIV coat proteins on proliferation are mediated by chemokine receptors.

### EXAMPLE 4

### HIV Coat Proteins can Reduce ERK Phosphorylation

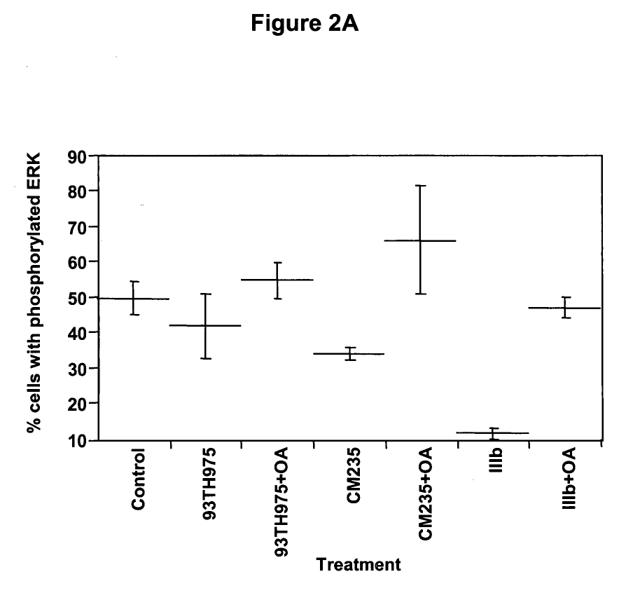

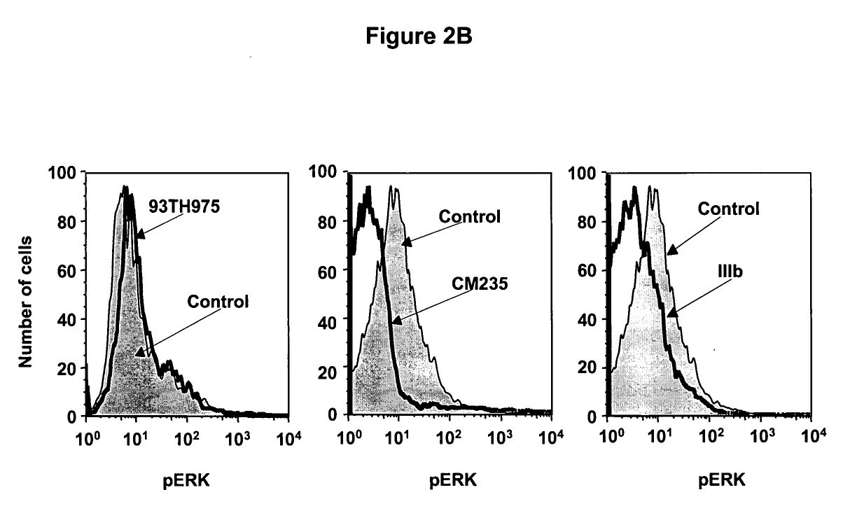

Proliferation of NPC is increased by signals that increase ERK phosphorylation (Learish et al. (2000) Develop. Brain Res. 122:97-109). Since chemokines have been shown to reduce ERK phosphorylation (Krathwohl et al., supra), the effect of HIV coat proteins on ERK phosphorylation was tested. NPC were incubated with a panel of HIV coat proteins and assayed to determine the fraction of cells with phosphorylated ERK. The CM235 and IIIb coat proteins inhibited ERK phosphorylation by 34% and 77% respectively (p<0.05 for both; FIGS. 2A and 2B). The 93TH975 coat protein showed a non-significant reduction of 19%. The phosphatase inhibitor okadaic acid blocked the inhibition of ERK phosphorylation that was induced by the CM235 and IIIb coat proteins (FIG. 2A). Thus, HIV coat proteins are able to activate a cellular phosphatase by signaling through chemokine receptors and reduce ERK phosphorylation, leading to reduced NPC proliferation.

### EXAMPLE 5

### In Vivo Effects of HIV Coat Proteins on NPC Proliferation

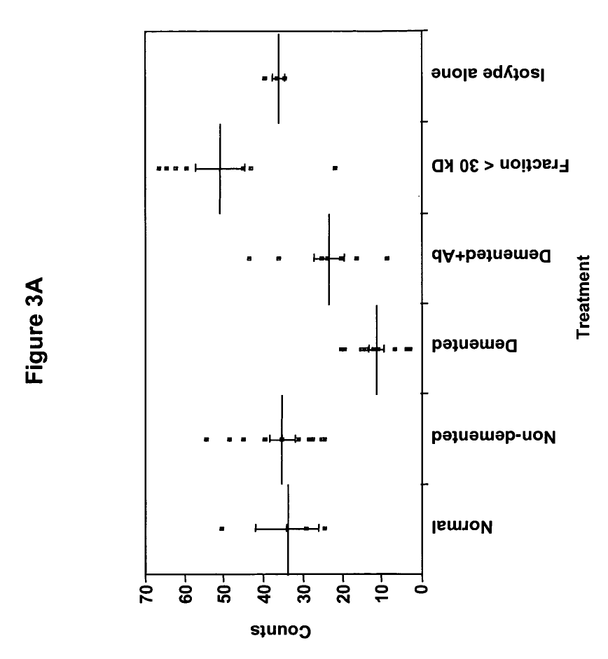

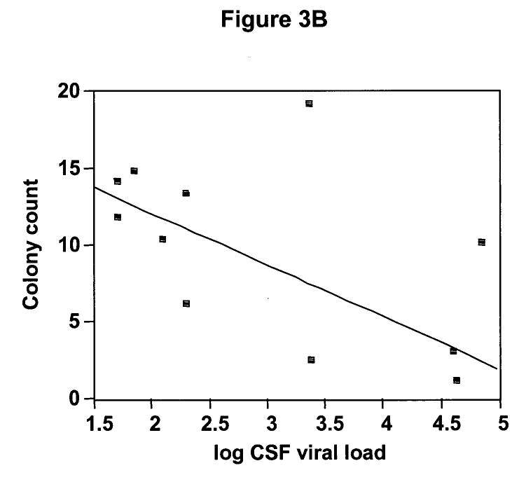

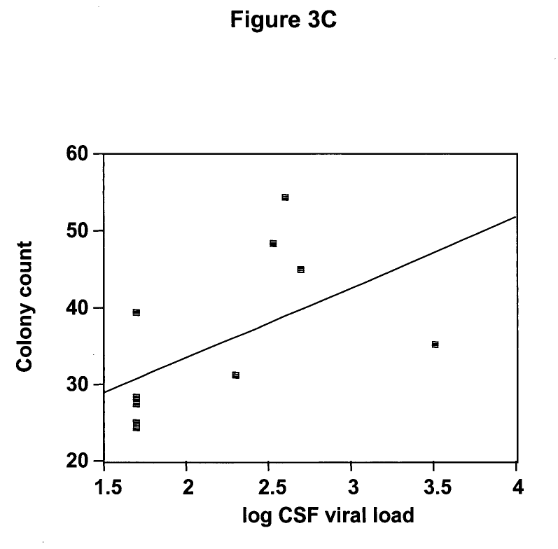

If HIV coat proteins play a role in vivo in regulating NPC proliferation, they should be found in sufficient quantities near NPC in order to have an effect. The only way to conveniently sample the central nervous system in living adults is by studying CSF. Although limitations exist when sampling CSF (Nath (2002) J. Infect. Dis. 186(Suppl. 2):S193-198), to a first approximation coat proteins shed by HIV infected cells should ultimately find their way into the CSF. Thus, experiments were conducted to test the ability of CSF from both demented and non-demented patients with HIV infection to suppress proliferation of NPC and induce quiescence. A panel of CSF from 10 demented and 10 non-demented patients with HIV infection was incubated with isolated NPC. CSF from demented patients reduced the number of differentiated colonies of NPC by 67% as compared to the number of differentiated colonies present with CSF from HIV-negative patients (p<0.001; FIG. 3A). CSF from non-demented patients showed results similar to CSF from HIV-negative patients. One patient with CSF samples taken before and after the onset of dementia showed colony formation identical to non-demented patients before dementia onset (38.2 colonies versus 35.4 for non-demented patients, p=0.5), but showed reduced colony formation after dementia onset (14.6 colonies, p<0.01). The broadly neutralizing antibody against the V3 loop of the HIV coat protein, which prevented chemokine-receptor signaling by the IIIb and CM235 coat proteins, was incubated with CSF from demented patients to determine if the inhibitory effect on NPC was due to the coat protein. The inhibitory effect of CSF from demented patients was blocked by the antibody (p<0.05 for CSF with and without antibody; FIG. 3A). In contrast, an irrelevant antibody had no effect on proliferation.

A great many molecules and viral proteins have been suggested as mediators of the toxic effects of HIV infection on the brain (Kolson et al. (1998) Adv. Virus Res. 50:1-45). Nearly all of the suggested toxins, other than gp120, have molecular weights less than 30,000 Daltons. Thus, CSF was separated into two fractions using a size exclusion column: a low molecular weight fraction (<30 kD) and a high molecular weight fraction (>30 kD). The gp120 coat protein was removed by this process, while RANTES was not. The retained proteins, presumably gp120, were able to inhibit NPC proliferation. However, the low molecular weight fraction of CSF, which should have contained molecules such as arachidonic acid, quinolinic acid, nitric oxide, TAT, other chemokines (e.g., TNF-α and TGFβ, among others) failed to suppress proliferation of NPC (FIG. 3A). It is possible that both the HIV coat protein and other molecules act together to inhibit NPC proliferation in any given patient. This was suggested by data from two patients whose CSF was both partially neutralized by the V3 antibody (34.7 and 6.3 colonies with V3 antibody vs. 44.3 for control) and partially inhibited by the <30 kD fraction of CSF (17.7 and 35.3 colonies vs. 45.5 for control). The mean viral load in CSF from demented patients was not significantly higher than that of non-demented patients (2.78 log vs. 2.21 log, p=0.31). However, the CSF viral load in demented patients correlated negatively with the number of NPC colonies formed (r=−0.657, p=0.039; FIG. 3B). In contrast, the CSF viral load in non-demented patients showed no correlation with colony formation (r=0.1, p=0.7; FIG. 3C). In addition, the presence or absence of antiretroviral therapy had no effect on the number of colonies formed. These results suggested that the coat protein in CSF is the predominant mediator of suppression of NPC proliferation in the patient population tested.

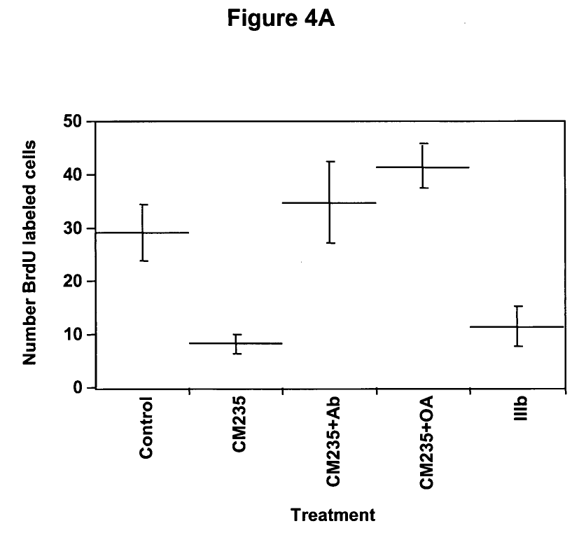

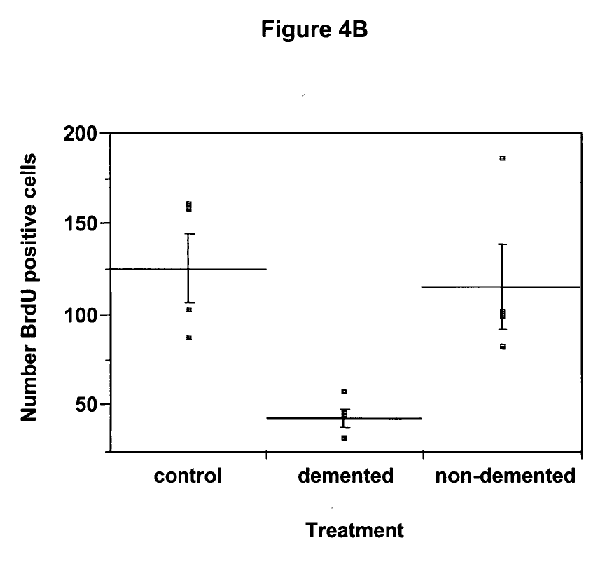

Because astrocytes and other cell types in the central nervous system can alter the effects of particular molecules on neurons (Oliet et al. (2001) Science 292:923-926; and Ye et al. (1998) Glia 22:237-248), the ability of chemokines and HIV proteins to affect NPC in the complex environment of the brain was tested. Differences likely exist between fetal and adult NPC, but NPC from both sources share many of the same markers (Palm et al. (2000) Mol. Brain Res. 78:192-195), including Musashil (Tonchev et al. (2003) Mol. Cell. Neurosci. 23:292-301). Thus, human hippocampal tissue was grown as slices in tissue culture to determine whether adult tissue would behave similarly when exposed to HIV. Cultures were treated with HIV coat proteins and pulsed with BrdU as a marker for proliferating NPC. As NPC are the only cells in this region of the brain that proliferate, BrdU positive cells can be assumed to be NPC (Kee et al. (2002) J. Neurosci. Meth. 115:97-105). HIV coat proteins CM235 and IIIb reduced proliferation of NPC by 75% and 70% respectively (p<0.001 for both; FIG. 4A). A broadly neutralizing antibody to the V3 region of gp120 blocked the effect of the CM235 protein. Thus, HIV coat proteins inhibit NPC proliferation in a more complex ex vivo system.

The ability of CSF from HIV patients to affect NPC was further tested in the hippocampal slice culture system. CSF from patients with and without dementia was incubated with hippocampal slices, which were then labeled with BrdU and harvested for immunohistochemistry. These studies showed that CSF from demented patients reduced the number of NPC by 66% (p<0.005; FIG. 4B). The number of NPC in slices cultured with CSF from non-demented patients was no different than the number observed in media controls. There was no difference in the number of apoptotic cells in demented versus non-demented CSF-treated slices (58.0 apoptotic cells/25 high-power fields vs. 60.5 cells, respectively; p=0.5). Therefore, CSF from demented patients specifically affects hippocampal tissue to reduce proliferation of NPC.

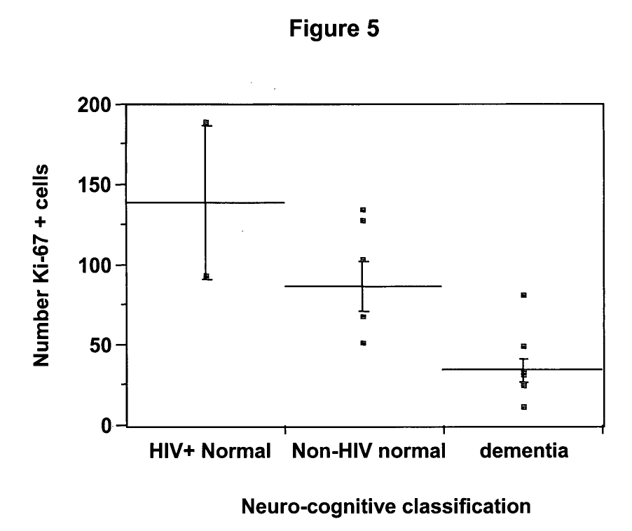

To determine if HIV patients with dementia exhibit reduced numbers of proliferating NPC, hippocampal tissue was obtained from autopsied patients with and without dementia. Tissue was stained for the proliferation marker Ki-67 in order to label NPC. HIV positive patients with dementia had 75% (p<0.005) fewer NPC than those without dementia (FIG. 5). There was no difference in the number of proliferating NPC between normal patients and non-demented HIV positive patients (p=0.2). Thus, patients with HIV dementia exhibit reduced numbers of proliferating NPC.

### EXAMPLE 6

### Heparan Sulfate can Block the Effects of HIV Coat Proteins on NPC

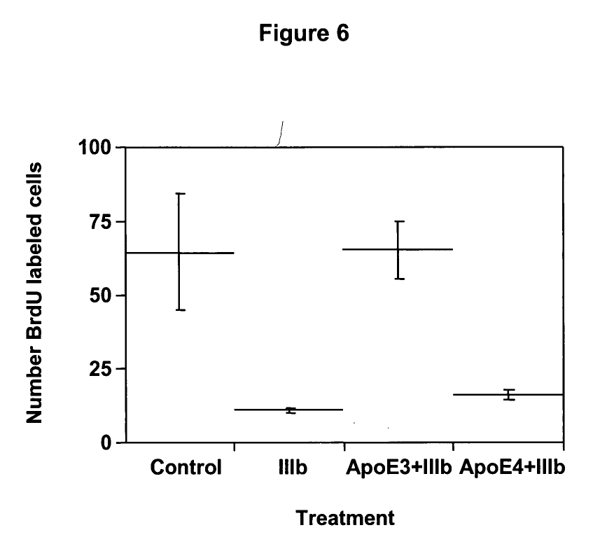

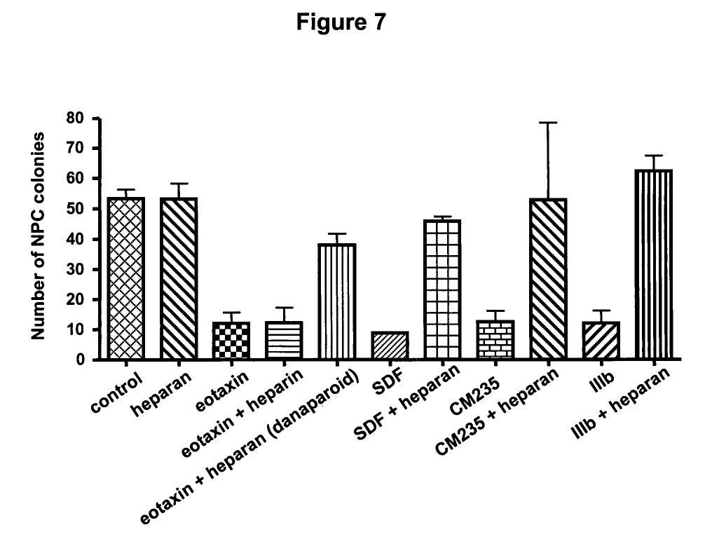

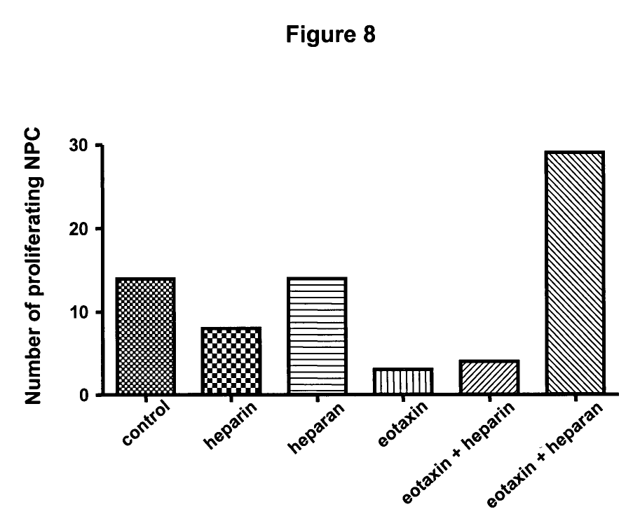

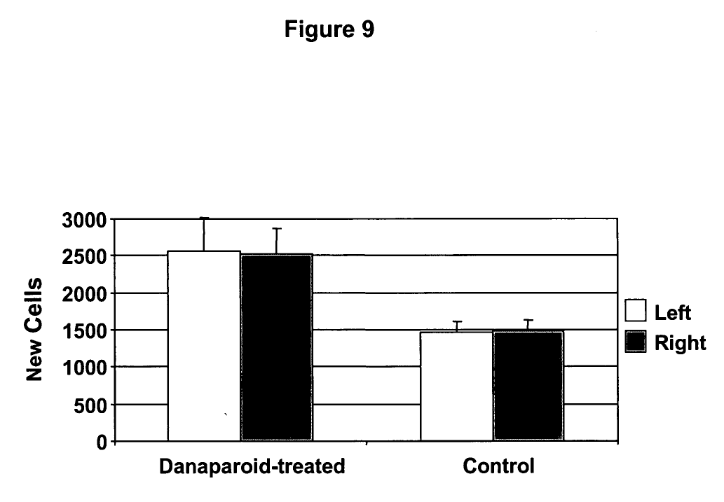

A risk factor for the development of HIV dementia is homozygosity for the apolipoprotein E4 genotype (Corder et al. (1998) Nat. Med. 4:1182-1184). In Herpes simplex virus infection, it has been speculated that apo E proteins compete with HSV for binding to HS, and that apo E4 cannot compete as efficiently as apo E3 (Itzhaki et al. (1998) Nat. Med. 4:1344). To determine whether apolipoproteins might compete with HIV coat proteins for binding to cell surface HS on NPC, human neurospheres were incubated with the IIIb coat protein, and the ability of recombinant apolipoproteins to block the inhibitory effect of the coat protein on NPC proliferation was tested. These experiments revealed that while 20 nM recombinant apo E3 protein prevented the IIIb coat protein from inhibiting NPC proliferation, the same amount of apo E4 protein did not (FIG. 6). Thus, apolipoproteins compete with HIV coat proteins for cell surface binding.

To determine whether HS (danaparoid) can prevent chemokines or HIV coat proteins from inhibiting NPC proliferation, cells were cultured with the chemokines eotaxin and SDF-1, with and without HS. Heparin was used for comparison purposes. HIV coat proteins from the CM235 and IIIb strains also were tested, since they activate the same co-receptors as eotaxin and SDF-1 respectively. These experiments revealed that HS blocked the inhibitory effects of both eotaxin and SDF-1 on NPC (FIG. 7). HS restored proliferation to control levels in these chemokine-treated cultures. In addition, HS blocked the inhibitory effects of both HIV coat proteins. Heparin had no effect. The ability of HS to block the inhibitory effects of chemokines on NPC also was tested within human hippocampal slices. Again, HS but not heparin blocked the inhibitory effect of chemokines (FIG. 8).

To determine whether HS could result in increased proliferation of NPC in vivo, rats were injected intravenously with saline or danaparoid, and proliferating cells were labeled with BrdU. Animals were sacrificed, and the number of BrdU-labeled cells was determined in the hippocampus of all animals. Treatment with danaparoid significantly increased the number of proliferating cells in both sides of the hippocampus, compared to control animals (FIG. 9). Thus, danaparoid may be useful for increasing NPC proliferation in vivo, and may be a useful therapy for dementia such as that induced by HIV.

The invention will be further described in the following examples, which do not limit the scope of the invention described in the claims.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a patient having a non-traumatic interneuron pathology, said method comprising administering to said patient a heparinoid, wherein said heparinoid is administered in an amount effective to reduce the symptoms of said non-traumatic interneuron pathology.

2. A method for treating a mammal, said method comprising administering to said mammal a heparinoid, wherein said heparinoid is administered in an amount effective to stimulate proliferation of NPC, and said method further comprising monitoring the cognitive function of said mammal.

3. The method of claim 2, further comprising adjusting the amount of heparinoid administered to said mammal based on said monitoring.

4. A method for treating dementia in a mammal in need thereof, said method comprising administering to said mammal an effective dose of a heparinoid.

5. The method of claim 4, wherein said mammal is a human.

6. The method of claim 4, wherein said mammal is HIV positive.

7. The method of claim 4, wherein said mammal has AIDS.

8. The method of claim 4, wherein said mammal is diagnosed with Alzheimer's disease.

9. The method of claim 4, wherein said heparinoid is heparan sulfate.

10. The method of claim 9, wherein said heparan sulfate is at least 70 percent pure.

11. The method of claim 9, wherein said heparan sulfate is at least 90 percent pure.

12. The method of claim 9, wherein said heparan sulfate is at least 99 percent pure.

13. The method of claim 9, wherein said heparan sulfate is in combination with chondroitin sulfate.

14. The method of claim 4, wherein said heparinoid is danaparoid.

15. A method for preventing dementia in a mammal, said method comprising administering a heparinoid to said mammal.

16. The method of claim 15, wherein said mammal is a human.

17. The method of claim 15, wherein said mammal is HIV positive.

18. The method of claim 15, wherein said mammal has AIDS.

19. The method of claim 15, wherein said mammal is diagnosed with Alzheimer's disease.

20. The method of claim 15, wherein said heparinoid is heparan sulfate.

21. The method of claim 20, wherein said heparan sulfate is at least 70 percent pure.

22. The method of claim 20, wherein said heparan sulfate is at least 90 percent pure.

23. The method of claim 20, wherein said heparan sulfate is at least 99 percent pure.

24. The method of claim 20, wherein said heparan sulfate is in combination with chondroitin sulfate.

25. The method of claim 15, wherein said heparinoid is danaparoid.

26. A method for treating memory loss in a mammal, said method comprising administering to said mammal an effective dose of a heparinoid.

27. A method for preventing memory loss in a mammal, said method comprising administering a heparinoid to said mammal.

28. A method for treating spinal cord injury in a mammal, said method comprising administering a heparinoid to said mammal.
